(19) United States
(12) Patent Application Publication
(10) Pub. No.: US 2022/0125324 A1
(43) Pub. Date: Apr. 28, 2022

(54) SYSTEM AND METHOD FOR DETERMINING A BLOOD FLOW CHARACTERISTIC

(71) Applicant: University of Louisville Research Foundation, Inc., Louisville, KY (US)

(72) Inventors: ROBERT ERIC BERSON, Louisville, KY (US); SHAHAB GHAFGHAZI, Louisville, KY (US); JAVAD HASHEMI, Louisville, KY (US)

(21) Appl. No.: 17/428,086

(22) PCT Filed: Feb. 10, 2020
(86) PCT No.: PCT/US20/17435

§ 371 Date: Aug. 3, 2021

Related U.S. Application Data
(60) Provisional application No. 62/803,636, filed on Feb. 11, 2019.

(51) Int. Cl. A61B 5/029; A61B 5/02; A61B 5/021; A61B 5/024; A61B 5/00; G16H 50/20; G16H 30/40; G16H 50/30

(52) U.S. Cl. CPC A61B 5/029; A61B 5/02028; A61B 5/021; G16H 50/30; A61B 5/7264; G16H 50/20; G16H 30/40; A61B 5/024

(57) ABSTRACT

A non-invasive method of assessing a coronary stenosis or other blockage in an artery or other vasculature is based on determination of a blood flow characteristic. In some embodiments, the blood flow characteristic is a fractional flow reserve determined using a statistical correlation of experimentally determined physiological factors and anatomical factors. In other embodiments, the blood flow characteristic is a blood flow rate determined using machine learning techniques. In further embodiments, the blood flow rate determined using machine learning techniques is a physiological factor used in determining the fractional flow reserve.

This application claims the benefit of U.S. provisional patent application Ser. No. 62/803,636, filed 11 Feb. 2019, for SYSTEM AND METHOD FOR DETERMINING FRACTIONAL FLOW RESERVE THROUGH A CORONARY STENOSIS, incorporated herein by reference.

### GOVERNMENT LICENSE RIGHTS

This invention was made with government support under Grant ID OGMB141515L1 awarded by the National Institutes of Health and under Grant ID 1355438 awarded by the National Science Foundation. The government has certain rights in the invention.

### FIELD OF THE INVENTION

A non-invasive method of assessing a coronary stenosis or other blockage in an artery or other vasculature is based on determination of a blood flow characteristic. In some embodiments, the blood flow characteristic is a fractional flow reserve determined using a statistical correlation of experimentally determined physiological factors and anatomical factors. In other embodiments, the blood flow characteristic is a blood flow rate determined using machine learning techniques. In further embodiments, the blood flow rate determined using machine learning techniques is a physiological factor used in determining the fractional flow reserve.

### BACKGROUND

The origin of cardiac events, such as myocardial infarction and aneurysm, are attributed to various hemodynamic factors, such as shear stress of regions of stagnant flow within the coronary arteries or other vasculature. In the United States, more than one million invasive coronary angiography (ICA) procedures are performed every year in patients who present with chest pain or are known to have stable coronary artery disease (CAD). The goal of the ICA procedure is to determine if there is any significant blockage (stenosis) that limits blood flow to the heart muscle in the coronary arteries. Almost half of ICA procedures culminate in stent placement in coronary arteries in order to relieve the blockage of blood flow. The cardiologist or other medical professional performing an ICA procedure determines the significance of the stenosis by one of two methods: (i) by visually estimating the degree of stenosis (“eyeballing” the stenosis), which is the routine practice and is performed for the majority of patients, or (ii) by invasively measuring fractional flow reserve (FFR). In this regard, FFR is defined as the ratio of the mean blood pressure downstream of the stenosis to the mean blood pressure upstream from the stenosis; in short, it is a measure of pressure differential across the stenosis. Normal FFR is 1 and an FFR<0.8 is considered hemodynamically significant. Invasively-measured FFR (i-FFR) is considered the more accurate and effective of the two described methods for determining the significance of a stenosis. However, i-FFR is only performed in 10-20% of patients in the United States because it is invasive, expensive, and time-consuming, and it also requires more radiation and contrast exposure than visual estimation of the stenosis.

As an alternative, efforts have been made to determine FFR though non-invasive methods. For example, a computer system can be configured to receive patient-specific data regarding a geometry of the heart and vasculature of a patient, such that a three-dimensional model can be created that represents at least a portion of the heart and/or vasculature. The computer system is further configured to create a physics-based model relating to a pressure (or other blood flow characteristic), and the computer system can then noninvasively determine a virtual FFR (v-FFR) based on the three-dimensional model and the physics-based model. Specifically, the computer system determines pressure loss across a stenosis or other blockage. Relevant United States patents and publications in the field of methods to determining v-FFR include U.S. Pat. Nos. 8,315,813, 9,189,600, and 9,339,200, and U.S. Patent Publication Nos. 2015/0302139 and 2016/0066861. However, the determination of the v-FFR requires significant computing resources.

### SUMMARY

The present disclosure is directed to a non-invasive method and system for assessing a coronary stenosis based directly on physiological and anatomical factors of a patient without the need for creation of a physics-based model of the vasculature or computational modeling blood flow in the vasculature. One of these factors is blood flow rate. While investigations have modeled blood flow rate as a fixed value, the present invention generates a mathematical estimate using one of a plurality of equations, the equation selected based on the type of vasculature, and using anatomical features of the vasculature as inputs. This method of generating a blood flow rate provides increased accuracy over fixed value models, which in turn results in improved non-invasive, non-computer modeled assessments of coronary stenosis based on blood flow rate and other physiological and anatomical factors.

It will be appreciated that the various systems and methods described in this summary section, as well as elsewhere in this application, can be expressed as a large number of different combinations and subcombinations. All such useful, novel, and inventive combinations and subcombinations are contemplated herein, it being recognized that the explicit expression of each of these combinations is unnecessary.

### BRIEF DESCRIPTION OF THE DRAWINGS

A better understanding of the present invention will be had upon reference to the following description in conjunction with the accompanying drawings.

FIG. 1A is a schematic of an eccentric stenosis.

FIG. 1B is a schematic of a concentric stenosis.

FIG. 2 is a chart comparing FFR determined statistically using the disclosed method and FFR determined using clinical techniques for a sample population of 69 patients.

FIG. 3 is chart comparing predicted and clinically determined volumetric blood flow rates.

### DETAILED DESCRIPTION OF THE PREFERRED EMBODIMENTS

In some embodiments, FFR is determined using a statistical correlation of known physiological and anatomical factors. Physiological factors include blood pressure as inlet boundary condition, blood flow rate as outlet boundary condition, and heart rate, blood viscosity and blood density. Anatomical factors include the diameter of the coronary artery, the length of the coronary segment modeled, the percent stenosis, stenosis length, the position of stenosis relative to coronary segment modeled, and the stenosis shape (that is, whether the stenosis is concentric or eccentric). Physiological factor data may obtained using standard techniques known in the art. Anatomical factor data may be obtained by standard imaging techniques, such as angiogram or CT scan. In some embodiments, anatomical factor data is obtained from analysis of a single angiogram image without creating a three-dimensional model of the vasculature. In other embodiments, anatomical factor data is obtained by obtaining two angiographic images, one image taken at a 30 degree angle to the other image, using the two images to create a three-dimensional model of the vasculature, and measuring the model to determine the desired anatomical factor data. In further embodiments, a greater number of angiographic images may be used to create a three-dimensional model of the vasculature.

Experimental design was used to determine which factors and interaction between factors contributed significantly to FFR. Eleven factors were initially tested with the Plackett-Burman design (Table 1). Placket-Burman designs are one means to determine the dependency of a measured quantity (i.e., FFR) on a plurality of factors. Identification and elimination of non-significant factors reduces computational and experimental effort in statistically determining FFR. FFR was determined by computational fluid dynamics (CFD) in model arteries created with the desired anatomical features to control the upper and lower range of the factors. Analysis of variance (ANOVA) showed a high variance coefficient (R²) value of 0.928. This analysis demonstrated that percent stenosis, diameter of coronary artery, blood flow rate, and stenosis shape had the most significant effect on FFR (p<0.05). In some embodiments, the factors of stenosis position and viscosity are also utilized, as they were determined to have a significance close, but not within, p<0.05. Stenosis position refers to the position of the stenosis along the vasculature of interest (i.e., 30% is more proximal, 50% is centered, and 70% is more distal).

To clarify, a v-FFR was determined using CFD based on a set of model arteries and this v-FFR was used to identify statistically significant anatomical and physiological factors for the novel system and method for diagnosing coronary stenosis, but CFD is not necessary in the practice of the system and method itself. As discussed below, a statistical FFR may be calculated based on the anatomical and physiological factors without computational modeling of vasculature structure or blood flow.

TABLE 1
The 11 factors tested by Plackett-Burman
design and upper and lower ranges.

| | Factors | Low Level | High Level |
|---|---|---|---|
| A | Blood Pressure (mmHg) | 80 | 140 |
| B | Blood Flow Rate (Kg/s) | 0.001 | 0.006 |
| C | Stenosis (%) | 65 | 70 |
| D | Length of Stenosis (mm) | 10 | 30 |
| E | Diameter of Artery (mm) | 2 | 6 |
| F | Length of Artery (mm) | 30 | 80 |
| G | Stenosis Position (%) | 30 | 70 |
| H | Stenosis Shape | Eccentric | Concentric |
| J | Heart Rate | 60 | 140 |
| K | Density (Kg/m³) | 1040 | 1080 |
| L | Viscosity (Kg/m/s) | 0.003 | 0.006 |

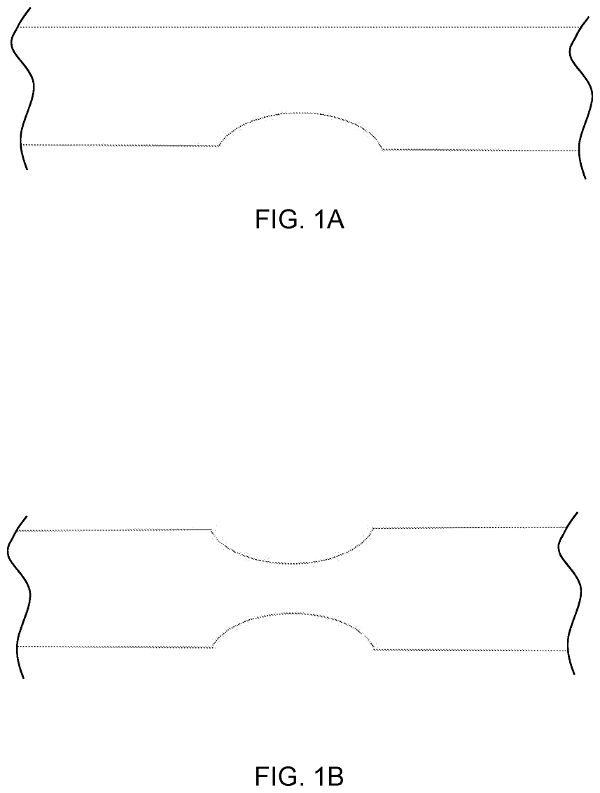

The Box-Behnken design was then performed with the six factors determined to be significant: percent stenosis, diameter of coronary artery, stenosis position, blood flow rate, viscosity, and stenosis shape (Table 2). Predicting FFR is highly complex and involves many factors and interactions. FFR responded nonlinearly with changing levels in coronary artery diameter and stenosis percentage, so the coronary diameter and stenosis percentage were divided into two sections for each factor. Diameter was tested as either 2-4 mm or 4-6 mm. Stenosis was tested as 40-60% or 60-80%, calculated as a ratio of the diameter of the stenosis to the diameter of the artery. The four Box-Behnken designs were tested according to Table 2. The statistical analysis for Box-Behnken designs showed that percent stenosis, diameter of artery and blood flow rate were significant for all designs. Table 3 shows the statistical model for each design; R² was 0.952, 0.940, 0.875 and 0.921 for designs from 1 to 4, respectively. The significant factors for the designs and significant interactions appear in Table 3, and separate models are provided for concentric and eccentric stenosis shape for each design. As shown in FIG. 1A, an eccentric stenosis is asymmetrical about the long axis of the vasculature of interest. As shown in FIG. 1B, a concentric stenosis is substantially symmetrical about the long axis of the vasculature of interest. Based on the Box-Behnken design, the statistical model was determined to be significant at p<0.05.

TABLE 2
Box-Behnken designs for six factors: (A) first design, (B)
second design, (C) third design, and (D) fourth design.

| | Factors | −1 | +1 |
|---|---|---|---|
| (A) | | | |
| A | Stenosis (%) | 40 | 60 |
| B | Diameter of Artery (mm) | 2 | 4 |
| C | Stenosis Position (%) | 0.25 | 0.75 |
| D | Blood Flow Rate (Kg/s) | 0.001 | 0.006 |
| E | Viscosity (Kg/m · s) | 0.003 | 0.006 |
| F | Stenosis Shape | Eccentric | Concentric |
| (B) | | | |
| A | Stenosis (%) | 60 | 80 |
| B | Diameter of Artery (mm) | 2 | 4 |
| C | Stenosis Position (%) | 0.25 | 0.75 |
| D | Blood Flow Rate (Kg/s) | 0.001 | 0.006 |
| E | Viscosity (Kg/m · s) | 0.003 | 0.006 |
| F | Stenosis Shape | Eccentric | Concentric |
| (C) | | | |
| A | Stenosis (%) | 40 | 60 |
| B | Diameter of Artery (mm) | 4 | 6 |
| C | Stenosis Position (%) | 0.25 | 0.75 |
| D | Blood Flow Rate (Kg/s) | 0.001 | 0.006 |
| E | Viscosity (Kg/m · s) | 0.003 | 0.006 |
| F | Stenosis Shape | Eccentric | Concentric |
| (D) | | | |
| A | Stenosis (%) | 60 | 80 |
| B | Diameter of Artery (mm) | 4 | 6 |
| C | Stenosis Position (%) | 0.25 | 0.75 |
| D | Blood Flow Rate (Kg/s) | 0.001 | 0.006 |
| E | Viscosity (Kg/m · s) | 0.003 | 0.006 |
| F | Stenosis Shape | Eccentric | Concentric |

TABLE 3
Statistical Models of the Box-Behnken designs.

| Design | Diameter | Stenosis | | | |
|---|---|---|---|---|---|
| Number | (mm) | (%) | FFR (Eccentric) | FFR (Concentric) | R² |
| 1 | 2-4 | 40-60 | +10.05991 | +13.48504 | 0.9521 |
| | | | −0.11815 * A | −0.18128 * A | |
| | | | −4.11167 * B | −5.58096 * B | |
| | | | −2457.30065 * D | −2257.06055 * D | |
| | | | −321.18849 * E | −321.18849 * E | |
| | | | +0.040320 * A * B | +0.067925 * A * B | |
| | | | +51.34811 * A* D | +38.95046 * A * D | |
| | | | +603.12073 * B * D | +720.12589 * B * D | |
| | | | +37.06076 * B * E | +37.06076 * B * E | |
| | | | −4.31850E−004 * A\hat{ }2 | −4.31850E−004 * A\hat{ }2 | |
| | | | +0.82232 * B\hat{ }2 | +0.82232 * B\hat{ }2 | |
| | | | +20732.26382 * E\hat{ }2 | +20732.26382 * E\hat{ }2 | |
| | | | +5.24695E−004 * A\hat{ }2 * B | +5.24695E−004 * A\hat{ }2 * B | |
| | | | −0.46041 * A\hat{ }2 * D | −0.46041 * A\hat{ }2 * D | |
| | | | −0.014610 * A * B\hat{ }2 | −0.014610 * A * B\hat{ }2 | |
| | | | −88.49070 * B\hat{ }2 * D | −88.49070 * B\hat{ }2 * D | |
| 2 | 2-4 | 60-80 | +116.05746 | +114.05840 | 0.9395 |
| | | | −2.57199 * A | −2.55750 * A | |
| | | | −15.95686 * B | −15.88458 * B | |
| | | | −242.43544 * C | −233.41467 * C | |
| | | | −449.78766 * D | +526.06099 * D | |
| | | | +0.10214 * A * B | +0.10214 * A * B | |
| | | | +5.75512 * A * C | +5.75512 * A * C | |
| | | | −12.55292 * A * D | −76.11972 * A * D | |
| | | | +23.84844 * B * C | +23.84844 * B * C | |
| | | | +212.50724 * B * D | +750.10112 * B * D | |
| | | | +2351.94761 * C * D | +4593.67314 * C * D | |
| | | | +0.016305 * A\hat{ }2 | +0.016305 * A\hat{ }2 | |
| | | | +1.40871 * B\hat{ }2 | +1.40871 * B\hat{ }2 | |
| | | | +7.54506 * C\hat{ }2 | −5.65765 * C\hat{ }2 | |
| | | | −0.041305 * A\hat{ }2 * C | −0.041305 * A\hat{ }2 * C | |
| | | | −3.89536 * B\hat{ }2 * C | −3.89536 * B\hat{ }2 * C | |
| | | | −2395.11944 * C\hat{ }2 * D | −2395.11944 * C\hat{ }2 * D | |
| 3 | 4-6 | 40-60 | +32517.67617 | +27360.22119 | 0.875 |
| | | | −601.02406 * A | −492.88781 * A | |
| | | | −1147.81925 * B | −888.86050 * B | |
| | | | −44669.12119 * C | −34312.60869 * C | |
| | | | −4.25716E+005 * D | −3.14858E+005 * D | |
| | | | −3200.33333 * E | −2.34648E+005 * E | |
| | | | +18.49937 * A * B | +18.49937 * A * B | |
| | | | +1421.59200 * A * C | +1200.62800 * A * C | |
| | | | −1996.50000 * A * D | −14387.50000 * A * D | |
| | | | +97040.50000 * B * D | +97040.50000 * B * D | |
| | | | −4.06800E+006 * D * E | +7.23867E+007 * D * E | |
| | | | +4.24878 * A\hat{ }2 | +4.24878 * A\hat{ }2 | |
| | | | +18806.78494 * C\hat{ }2 | +18806.78494 * C\hat{ }2 | |
| | | | −10.48138 * A\hat{ }2 * C | −10.48138 * A\hat{ }2 * C | |
| | | | −375.68267 * A * C\hat{ }2 | −375.68267 * A * C\hat{ }2 | |
| 4 | 4-6 | 60-80 | −1.34456E+005 | −1.56771E+005 | 0.9206 |
| | | | +6535.32525 * A | +9309.57615 * A | |
| | | | +5029.18868 * B | −20586.05374 * B | |
| | | | −2.29288E+007 * D | −2.73546E+007 * D | |
| | | | +2.70765E+005 * E | +3.81717E+006 * E | |
| | | | −1169.83309 * A * B | −806.99181 * A * B | |
| | | | +7.36593E+005 * A* D | +7.36593E+005 * A * D | |
| | | | −4720.33333 * A* E | −59990.47867 * A * E | |
| | | | +1.34061 E+005 * B * D | +7.80911E+005 * B * D | |
| | | | −65.32143 * A\hat{ }2 | −99.50274 * A\hat{ }2 | |
| | | | +7269.37270 * B\hat{ }2 | +7269.37270 * B\hat{ }2 | |
| | | | +16.53660 * A\hat{ }2 * B | +16.53660 * A\hat{ }2 * B | |
| | | | −5990.60802 * A\hat{ }2 * D | −5990.60802 * A\hat{ }2 * D | |
| | | | −110.47687 * A * B\hat{ }2 | −110.47687 * A * B\hat{ }2 | |

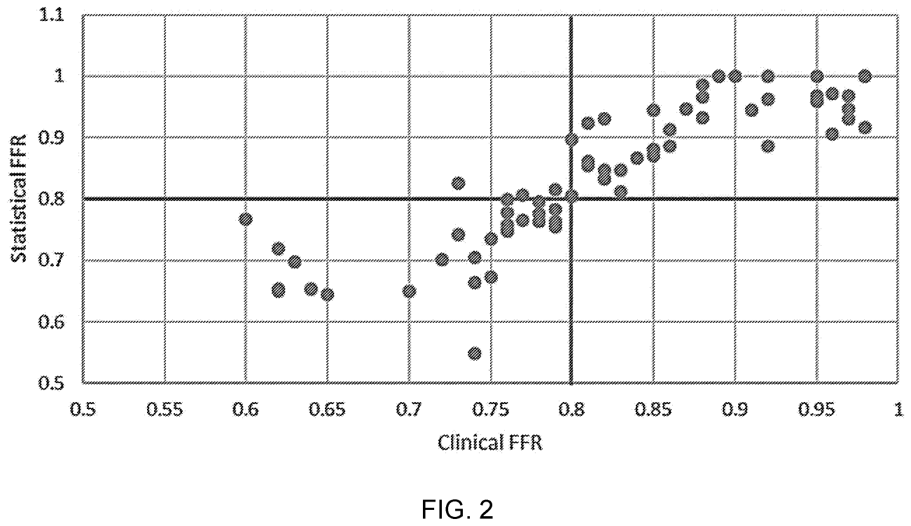

The eight equations provided in Table 3 are used to produce a statistically-determined FFR (s-FFR), a unitless value, based on physiological and anatomical factors. A FFR less than 0.8 is considered hemodynamically significant and indicates a coronary stenosis that poses a potential risk to patient health. FIG. 2 displays a comparison of s-FFRs determined using the disclosed method and FFR values determined using clinical methods for the same sample population, showing the efficacy of the disclosed method.

Percent stenosis, artery diameter, stenosis position, viscosity and stenosis shape (Factors A, B, C, E, and F in Table 2) are determined from analysis of the patient's angiogram image(s). Factor D, blood flow rate, cannot currently be determined by a simple non-invasive diagnostic technique. Blood flow rate may be considered in either terms of mass (as discussed above) or in terms of volume, and can easily be converted between the two (dividing mass flow by density=1045 kg/m³ yields the volumetric flow rate). Blood flow rate proximal to a coronary stenosis has been identified as an important inlet boundary condition to determine v-FFR without the need to model blood flow throughout the entire coronary tree. However, determining this inlet flow rate is problematic because it requires knowing the left ventricle volume, which is not obtainable from coronary angiography. One solution to the problem has been the use of a fixed volume flow rate in all patients (e.g., 1 ml/min under baseline conditions or 3 ml/min under hyperemic conditions). However, there is a significant variation in volume flow rate from patient to patient so this solution sacrifices accuracy. Attempts to determine individualized flow rates have proved to be time consuming and/or require invasive techniques.

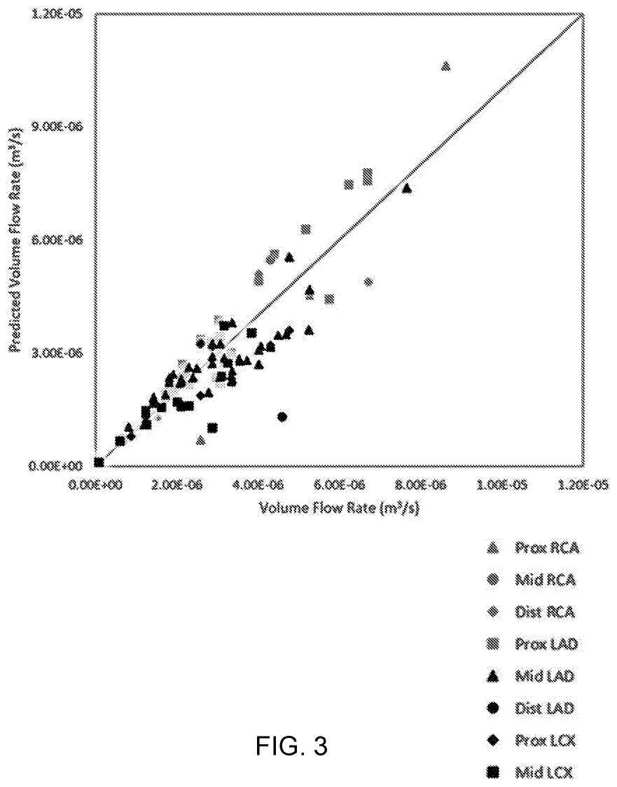

Here, a multiple linear regression approach was employed to determine coronary volume flow rate for patients undergoing coronary angiography. The actual inlet blood volume flow rate proximal to stenotic coronary segments was determined clinically for a sample population and correlated with anatomical images obtained from the same population. The anatomical factors determined to be significant in affecting inlet blood volume flow rate by this approach are: coronary segment type (A), inlet diameter of the segment (B), stenosis diameter (C), stenosis percentage (D), inlet area of the segment (E), and stenosis area (F). CFD modeling suggests coronary segment type (i.e., factor A) is the most significant determinant of inlet blood volume flow rate. The clustering method was used to divide coronary arteries into proximal, mid, and distal segments. The sample population was divided into subgroups based on specific segment types, and multiple linear regression then used with other factors B-F for each subgroup. Table 4 shows the machine learning models for flow rate generated for each segment type and the accuracy thereof. FIG. 3 graphically demonstrates the accuracy of this method in predicting coronary inlet volume blood flow rate for each segment, with clinical based inlet blood volume flow rate as the reference. For clarification, factors A-F significant for determining blood flow rate are distinct from factors A-F significant for determining SFV, although blood flow rate is one of the factors used for determining FFV.

TABLE 4
Machine Learning Model for Volumetric Blood Flor Rate Clustered by Segment Type.

| | Number | Inlet | | | |
|---|---|---|---|---|---|
| Segment | of | Diameter | Validation | Regression | |
| Type | Patients | Range (mm) | R² | Model R² | Blood Flow Rate Estimation |
| Prox RCA | 4 | 3.5-5 | 0.989 | 1 | =1.474E−005 |
| (right coronary | | | | | −1.890E−007*C*D |
| artery) | | | | | +1.247E−009*D*E |
| | | | | | +2.590E−007*F\hat{ }2 |
| Mid RCA | 6 | 2.8-3.7 | 0.974 | 0.997 | =−5.465E−005 |
| | | | | | +9.855E−007*D |
| | | | | | +5.808E−006*C\hat{ }2 |
| | | | | | −3.009E−007*E\hat{ }2 |
| | | | | | +2.960E−006*F\hat{ }2 |
| Dist RCA | 4 | 3.8-4 | 0.986 | 1 | =3.529E−006 |
| | | | | | −3.576E−006*C |
| | | | | | +1.217E−008*D |
| | | | | | +5.318E−007*F\hat{ }2 |
| Prox LAD | 21 | 3.5-5 | 0.942 | 0.954 | =−4.975E−005 |
| (left anterior | | | | | +1.495E−006*D |
| descending | | | | | −3.680E−006*E |
| coronary | | | | | +1.509E−007*B*D |
| artery) | | | | | −1.331E−006*C*D |
| | | | | | +5.040E−007*D*F |
| | | | | | −9.890E−007*E*F |
| | | | | | −2.680E−006*B\hat{ }2 |
| | | | | | +2.853E−005*C\hat{ }2 |
| | | | | | +3.069E−007*E\hat{ }2 |
| | | | | | −3.078E−006*F\hat{ }2 |
| Mid LAD | 42 | 2-4.5 | 0.852 | 0.82 | =−1.548E−005 |
| | | | | | +2.857E−005*C |
| | | | | | +2.798E−007*D |
| | | | | | +5.730E−007*E |
| | | | | | +2.504E−006*F |
| | | | | | −1.522E−007*B*D |
| | | | | | −1.617E−007*C*D |
| | | | | | +7.556E−007*C*E |
| | | | | | −4.781E−008*D*F |
| | | | | | +6.764E−008*E*F |
| | | | | | +2.100E−006*B\hat{ }2 |
| | | | | | −1.450E−005*C\hat{ }2 |
| | | | | | −7.612E−008*E\hat{ }2 |
| | | | | | +1.048E−007*F\hat{ }2 |
| Dist LAD | 3 | 2.8-3.8 | 0.933 | 0.905 | =2.226E−006 |
| | | | | | +2.303E−008*C*D |
| | | | | | +1.533E−009*D*E |
| | | | | | −1.322E−008*D*F |
| | | | | | −6.302E−010*D\hat{ }2 |
| | | | | | −4.095E−008*F\hat{ }2 |
| Prox LCX | 7 | 2.8-4 | 0.992 | 1 | =1.156E−005 |
| (left | | | | | −8.360E−008*D |
| circumference | | | | | +4.890E−007*E |
| branches) | | | | | +6.593E−008*B*D |
| | | | | | −2.534E−007*C*D |
| | | | | | −1.002E 008*D*F |
| | | | | | +1.637E−007*F\hat{ }2 |
| MID LCX | 13 | 3.1-4 | 0.981 | 1 | =3.079E−005 |
| | | | | | −4.427E−005*B |
| | | | | | +1.874E−007*B*D |
| | | | | | +1.059E−006*C*D |
| | | | | | −1.511E−007*D*F |
| | | | | | +2.673E−006F\hat{ }2 |
| Total | 100 | | | | |

The generated equations show there is significant variation in inlet volume flow rate, based in part on the segment type containing the stenosis. The R² for seven of the eight segment types was between 0.9 and 1.0. The Mid LAD regression model had the lowest accuracy (0.82), likely due to this segment having the widest variation in inlet diameter.

This novel method of non-invasive determination of blood flow rate, specifically, blood flow rate proximal to stenotic coronary segments, allows for the determination of a s-FFR based on a plurality of anatomical and physiological factors, including said blood flow rate, using only anatomical images, and without constructing a 3D model of the coronary tree or use of computationally intensive CFD.

Various aspects of different embodiments of the present disclosure are expressed in paragraphs X1, X2, X3, and X4 as follows:

X1: One embodiment of the present disclosure includes a method for assessing a stenosis in a vasculature of interest, the method comprising obtaining at least one anatomical image of the vasculature of interest; determining at least one anatomical factor of the vasculature of interest based at least one anatomical image; determining at least one physiological factor of the vasculature of interest; calculating a statistical fractional flow reserve based on the at least one physiological factor and the at least one anatomical factor; and designating the stenosis as hemodynamically significant if the statistical fractional flow reserve is less than a predetermined value.

X2: Another embodiment of the present disclosure includes a method for determining the hemodynamic significance of a stenosis in a vasculature of interest, the method comprising obtaining at least one anatomical image of the vasculature of interest; determining at least one anatomical factor of the vasculature of interest based at least in part on the at least one anatomical image; determining at least one physiological factor of the vasculature of interest; calculating a statistical fractional flow reserve based on the at least one physiological factor and the at least one anatomical factor; and designating the stenosis as hemodynamically significant if the statistical fractional flow reserve is less than a predetermined value.

X3: A further embodiment of the present disclosure includes a method of determining fractional flow reserve in a blood vessel having a stenosis, the method comprising obtaining at least one anatomical image of the blood vessel; determining at least one anatomical factor of the blood vessel based at least in part on the at least one anatomical image; determining at least one physiological factor of the vasculature of interest; calculating a fractional flow reserve based on the at least one physiological factor and the at least one anatomical factor.

X4: Another embodiment of the present disclosure includes a non-invasive method for determining a blood flow rate in a blood vessel containing a stenosis, the method comprising obtaining at least one anatomical image of a blood vessel; determining at least two anatomical factors of the blood vessel based at least in part on the at least one anatomical image, wherein one of the at least two anatomical factors is a blood vessel segment type; selecting one of a plurality of equations, the selection based on the blood vessel segment type; calculating a blood flow rate using the selected equation using the anatomical factors as inputs into the selected equation.

Yet other embodiments include the features described in any of the previous paragraphs X1, X2, X3, or X4 as combined with one or more of the following aspects:

Wherein the at least one anatomical factor is at least one of percent stenosis, length of stenosis, diameter of artery, length of artery, stenosis position, and stenosis shape.

Wherein the stenosis shape is one of concentric and eccentric.

Wherein the at least one anatomical factor is at least one of percent stenosis, diameter of artery, stenosis position, and stenosis shape.

Wherein the at least one anatomical factor is at least two of percent stenosis, diameter of coronary artery, stenosis position, and stenosis shape.

Wherein the at least one anatomical factor is at least three of percent stenosis, diameter of coronary artery, stenosis position, and stenosis shape.

Wherein the at least one anatomical factor is percent stenosis, diameter of coronary artery, stenosis position, and stenosis shape.

Wherein the at least one physiological factor is at least one of blood pressure, blood flow rate, heart rate, blood density, and blood viscosity.

Wherein the at least one physiological factor is at least two of blood pressure, blood flow rate, heart rate, blood density, and blood viscosity.

Wherein the at least one physiological factor is at least three of blood pressure, blood flow rate, heart rate, blood density, and blood viscosity.

Wherein the at least one physiological factor is blood pressure, blood flow rate, heart rate, blood density, and blood viscosity.

Wherein the at least one physiological factor is at least one of blood flow rate and blood viscosity.

Wherein the at least one physiological factor is blood flow rate.

Wherein blood flow rate is calculated based on a machine learning model.

Wherein blood flow rate is calculated based on a plurality of factors, including at least one of segment type, segment inlet diameter, stenosis diameter, stenosis percentage, segment inlet area, and stenosis area.

Wherein blood flow rate is calculated based on a plurality of factors, including at least two of segment type, segment inlet diameter, stenosis diameter, stenosis percentage, segment inlet area, and stenosis area.

Wherein blood flow rate is calculated based on a plurality of factors, including at least three of segment type, segment inlet diameter, stenosis diameter, stenosis percentage, segment inlet area, and stenosis area.

Wherein blood flow rate is calculated based on segment type and at least one of segment inlet diameter, stenosis diameter, stenosis percentage, segment inlet area, and stenosis area.

Wherein blood flow rate is calculated based on segment type, segment inlet diameter, stenosis diameter, stenosis percentage, segment inlet area, and stenosis area.

Wherein the at least one anatomical factor is at least one of percent stenosis, length of stenosis, diameter of artery, length of artery, stenosis position, and stenosis shape, and wherein the at least one physiological factor is at least one of blood pressure, blood flow rate, heart rate, blood density, and blood viscosity.

Wherein the at least one anatomical factor is at least one of percent stenosis, diameter of artery, stenosis position, and stenosis shape, and wherein the at least one physiological factor is at least one of blood flow rate and blood viscosity.

Wherein the predetermined value is between 0.7 and 0.9.

Wherein the predetermined value is between 0.75 and 0.85.

Wherein the predetermined value is 0.8.

Wherein the at least one anatomical image is only one anatomical image.

Wherein said calculating comprises selecting one of a plurality of equations, the selection based on the at least one anatomical factor or the at least one physiological factor, and calculating the statistical fractional flow reserve using the selected equation.

Wherein the plurality of equations are generated using a machine learning model trained on anatomical images for different blood vessel segment types.

Wherein the plurality of equations are generated using a machine learning model trained on anatomical images for different blood vessel segment types for which blood flow rates were clincially determined.

Wherein the blood flow rate is one a plurality of factors used in calculating a statistical fractional flow reserve, and wherein blood vessel is determined to have a hemodynamically significant stenosis if the statistical fractional flow reserve is less than a predetermined value.

The foregoing detailed description is given primarily for clearness of understanding and no unnecessary limitations are to be understood therefrom for modifications can be made by those skilled in the art upon reading this disclosure and may be made without departing from the spirit of the invention.

What is claimed is:

1) A method for assessing a stenosis in a vasculature of interest, the method comprising:
obtaining at least one anatomical image of the vasculature of interest;
determining at least one anatomical factor of the vasculature of interest based at least one anatomical image;
determining at least one physiological factor of the vasculature of interest;
calculating a statistical fractional flow reserve based on the at least one physiological factor and the at least one anatomical factor; and
designating the stenosis as hemodynamically significant if the statistical fractional flow reserve is less than a predetermined value.

2) The method of claim 1, wherein the at least one anatomical factor is at least one of percent stenosis, length of stenosis, diameter of artery, length of artery, stenosis position, and stenosis shape.

3) The method of claim 2, wherein stenosis shape is one of concentric and eccentric.

4) The method of claim 1, wherein the at least one anatomical factor is at least one of percent stenosis, diameter of artery, stenosis position, and stenosis shape.

5) The method of claim 1, wherein the at least one anatomical factor is at least two of percent stenosis, diameter of coronary artery, stenosis position, and stenosis shape.

6) The method of claim 1, wherein the at least one physiological factor is at least one of blood pressure, blood flow rate, heart rate, blood density, and blood viscosity.

7) The method of claim 1, wherein the at least one physiological factor is at least one of blood flow rate and blood viscosity.

8) The method of claim 1, wherein the at least one physiological factor is blood flow rate.

9) The method of claim 8, wherein blood flow rate is calculated based on a machine learning model.

10) The method of claim 8, wherein blood flow rate is calculated based on a plurality of factors, including at least one of segment type, segment inlet diameter, stenosis diameter, stenosis percentage, segment inlet area, and stenosis area.

11) The method of claim 1, wherein the predetermined value is 0.8.

12) The method of claim 1, wherein said calculating comprises selecting one of a plurality of equations, the selection based on the at least one anatomical factor or the at least one physiological factor, and calculating the statistical fractional flow reserve using the selected equation.

13) A method for determining the hemodynamic significance of a stenosis in a vasculature of interest, the method comprising:
obtaining at least one anatomical image of the vasculature of interest;
determining at least one anatomical factor of the vasculature of interest based at least in part on the at least one anatomical image;
determining at least one physiological factor of the vasculature of interest;
calculating a statistical fractional flow reserve based on the at least one physiological factor and the at least one anatomical factor; and
designating the stenosis as hemodynamically significant if the statistical fractional flow reserve is less than a predetermined value.

14) The method of claim 13, wherein the predetermined value is 0.8.

15) The method of claim 13, wherein the at least one anatomical factor is at least one of percent stenosis, length of stenosis, diameter of artery, length of artery, stenosis position, and stenosis shape, and wherein the at least one physiological factor is at least one of blood pressure, blood flow rate, heart rate, blood density, and blood viscosity.

16) The method of claim 13, wherein the at least one anatomical factor is at least one of percent stenosis, diameter of artery, stenosis position, and stenosis shape, and wherein the at least one physiological factor is at least one of blood flow rate and blood viscosity.

17) The method of claim 13, wherein the at least one anatomical image is only one anatomical image.

18) A method of determining fractional flow reserve in a blood vessel having a stenosis, the method comprising:
obtaining at least one anatomical image of the blood vessel;
determining at least one anatomical factor of the blood vessel based at least in part on the at least one anatomical image;
determining at least one physiological factor of the vasculature of interest;
calculating a fractional flow reserve based on the at least one physiological factor and the at least one anatomical factor.

19) The method of claim 18, wherein the at least one physiological factor is blood flow rate.

20) The method of claim 18, wherein said calculating comprises selecting one of a plurality of equations, the selection based on the at least one anatomical factor or the at least one physiological factor, and calculating the statistical fractional flow reserve using the selected equation.

21) A non-invasive method for determining a blood flow rate in a blood vessel containing a stenosis, the method comprising:
obtaining at least one anatomical image of a blood vessel;
determining at least two anatomical factors of the blood vessel based at least in part on the at least one anatomical image, wherein one of the at least two anatomical factors is a blood vessel segment type;
selecting one of a plurality of equations, the selection based on the blood vessel segment type;
calculating a blood flow rate using the selected equation using the anatomical factors as inputs into the selected equation.

22) The method of claim 21, wherein the plurality of equations are generated using a machine learning model trained on anatomical images for different blood vessel segment types for which blood flow rates were clincially determined.

23) The method of claim 21, wherein the at least two anatomical factors include at least two of blood vessel segment type, segment inlet diameter, stenosis diameter, stenosis percentage, segment inlet area, and stenosis area.

24) The method of claim 21, wherein the blood flow rate is one a plurality of factors used in calculating a statistical fractional flow reserve, and wherein blood vessel is determined to have a hemodynamically significant stenosis if the statistical fractional flow reserve is less than a predetermined value.
